(19) United States
(12) Patent Application Publication
(10) Pub. No.: US 2009/0233312 A1
(43) Pub. Date: Sep. 17, 2009

(54) METHODS AND ASSAYS TO ASSESS CARDIAC RISK AND ISCHEMIA

(75) Inventors: Winton G. Gibbons, Winnetka, IL (US); Thomas F. Holzman, Libertyville, IL (US); Phillip A. Lefebvre, Lincolnshire, IL (US); Gregory W. Shipp, Hawthorn Woods, IL (US)

Correspondence Address: GREGORY T. PLETTA;Nanosphere, Inc., 4088 Commerical Avenue, Northbrook, IL, 60062-1829, US

(73) Assignee: Nanosphere, Inc., Northbrook, IL (US)

(21) Appl. No.: 12/249,903

(22) Filed: Oct. 10, 2008

Related U.S. Application Data
(60) Provisional application No. 60/998,457, filed on Oct. 10, 2007.

(51) Int. Cl. G01N 33/53

(52) U.S. Cl. 435/7.1

(57) ABSTRACT

The invention provides methods and apparatus to assess cardiac risk and ischemia by detecting or analyzing cardiac troponin levels. Also provided are methods to detect low levels of cardiac troponin in physiological fluid samples.

### CROSS-REFERENCE TO RELATED APPLICATIONS

The present application claims the benefit of the filing date of U.S. application Ser. No. 60/998,457, filed on Oct. 10, 2007, the disclosure of which is incorporated by reference herein.

### BACKGROUND

Cardiac troponin T (cTn) was introduced in the routine laboratory diagnostic work-up in 1989 to sensitively indicate acute myocardial ischaemia (MI). Primary causes of troponin T positivity include MI, unstable coronary heart disease, myocarditis, hypertrophic cardiomyopathy, uraemic cardiomyopathy, atrial fibrillation, congestive heart failure, hypotonia, hypovolaemia, percutaneous coronary intervention, and ASD closure. Primary causes of troponin I positivity include MI, unstable coronary heart disease, myocarditis, pericarditis, hypertrophic cardiomyopathy, tachycardia, atrial fibrillation, congestive heart failure, increased left ventricular mass, severe aortic valve disease, coronary vasospasm, cardiac contusion, cardiac tamponade, hypertensive crisis, implantable cardioverter defribrillator shocks, electrical cardioversion, percutaneous coronary intervention, ASD closure, radiofrequency ablation, cardiac transplantation, and pacemaker implantation.

The most frequency of causes for troponin T positivity when multiple causes are at issue include MI, atrial fibrillation, congestive heart failure, chronic renal insufficiency, percutaneous cardiac interventions, chronic obstructive pulmonary disease, tachycardia, acute stroke, electric cardioversion, dilated cardiomyopathy, increased left ventricular mass, aortic valve disease, and hypertensive crisis. However, by far the most common single cause for troponin T positivity is MI.

Current cardiac troponin assays report one concentration for any and all cardiac troponins having cTnI or cTnT isoforms and their serum carried, metabolized products at one time point per sample. Increasing target incubation time can increase the amount of target captured, and thus the potential signal generated. However, current clinical cTn protein detection assays rely on signal generation technologies that lack sufficient sensitivity to detect lower amounts of cTn that may be clinically relevant. To address this issue, these assays require higher initial target concentrations to produce a detectable signal or require assay times beyond a clinically relevant time frame (i.e., several hours). The state-of-the-art is to use point-of-care instruments for rapid diagnosis (a few minutes) of patients with extremely high cTn1 concentrations (e.g., >80 pg/mL), or laboratory-based instruments for slower (tens of minutes), more thorough diagnosis (e.g., down to about 10 pg/mL cTn1 concentrations) of less severe but critical cardiac events.

### SUMMARY OF THE INVENTION

The invention provides a method to determine a mammal or other test subject at risk of or suspected of having unstable angina or non-ST elevation myocardial infarction. The method includes detecting the amount of first complexes formed by contacting a first physiological fluid sample from a test subject at risk of or suspected of having acute coronary syndrome and a substrate having one or more moieties that specifically bind cardiac troponin, thereby detecting the concentration of troponin in the first physiological sample. The amount of the first complexes is compared with second complexes formed by contacting a second physiological sample from the test subject from a different time point with a substrate having the one or more moieties that specifically bind cardiac troponin. The rate of increase in cardiac troponin concentrations over time may be indicative of, for example, MI, unstable angina (UA) or non-ST elevation myocardial infarction (NSTEMI). In one embodiment, the test subject is a human. In one embodiment, the one or more moieties are monoclonal antibodies which are employed to capture, immobilize or detect cardiac troponin. In one embodiment, the one or more moieties are polyclonal antibodies, e.g., a composition comprising cardiac troponin-specific polyclonal antibodies is employed to capture, immobilize or detect cardiac troponin. In one embodiment, the one or more capture cardiac troponin-specific antibodies are specific for one epitope of troponin and the one or more detection cardiac troponin-specific antibodies bind more than one epitope of troponin. In one embodiment, the one or more capture cardiac troponin-specific antibodies bind more than one epitope of troponin and the one or more detection cardiac troponin-specific antibodies are specific for one epitope of troponin.

In one embodiment, the method provides for detection of cardiac troponin concentrations of 10 pg/mL or less, e.g., less than about 1 pg/mL, based on calibration to the independently established NIST troponin standard (National Institute of Standards and Technology; see www.nist.gov), e.g., concentrations of 2 fg/mL to 10 pg/mL, 2 fg/mL to 100 fg/mL or 10 fg/mL to 500 fg/mL. Such a sensitive assay allows for detection of a test subject that is not at risk of or having a cardiac event, e.g., not at risk of or having acute coronary syndrome, as well as distinguishing between acute coronary syndromes, such as UA and NSTEMI. For instance, elevated cardiac troponin levels that do not substantially increase over time (e.g., 2.0 fold) likely indicate chronic conditions, while a greater rate increase in cardiac troponin levels over time may be indicative of UA or NSTEMI. The time points for comparison may be minutes apart, e.g., 5, 10, 20, or 30 minutes apart, hours apart, e.g., 1, 2, 4, 6, or 8 hours apart, or one or more days apart, or any combination thereof. In one embodiment, the rate of increase of cardiac troponin levels in a test subject having NSTEMI is greater than the rate of increase of cardiac troponin levels in a test subject having UA. In one embodiment, the complexes are detected with one or more second moieties that specifically bind cardiac troponin linked to a detectable molecule, such as a nanoparticle, an oligonucleotide or barcode. In one embodiment, to enhance the detection of the detectable molecule, the signal generated by the detectable molecule can be amplified. For instance, a silver coating (deposition) on a gold nanoparticle bound to a complex on a substrate can amplify the signal generated by the presence of the gold nanoparticle when exposed to light and a fluorescently labeled primer that hybridizes to an oligo- or poly-nucleotide bound to a complex on a substrate can be employed to amplify nucleic acid sequences in the oligo- or poly-nucleotide by enzymatic processes.

In one embodiment, the invention provides a method to detect cardiac troponin concentrations of 10 pg/mL or less. The method includes detecting the amount of first complexes formed by contacting a first physiological fluid sample from a test subject and a solid substrate having one or more cardiac troponin-specific antibodies, thereby detecting the concentration of troponin in the first physiological sample, wherein cardiac troponin concentrations of 10 pg/mL or less are detectable. In one embodiment, the method includes comprising comparing the amount of the first complexes with second complexes formed by contacting a second physiological sample from the test subject from a different time point with a solid substrate having the one or more cardiac troponin-specific antibodies, wherein the rate of increase in the amount of cardiac troponin over time is indicative of acute coronary syndrome in the test subject.

Also provided is a method to detect cardiac troponin levels in a physiological sample. The method includes providing a mixture comprising a physiological fluid sample from a test subject and a solid substrate having one or more moieties that specifically bind cardiac troponin so as to form a first complex. That complex is contacted with one or more different moieties that specifically bind cardiac troponin and are attached to a detectable molecule. The presence or amount of the detectable molecule is detected or determined, thereby detecting or determining cardiac troponin levels. In one embodiment, the method provides for detection of cardiac troponin concentrations of less than 10 pg/mL, e.g., less than 1 pg/mL based on calibration to the independently established NIST troponin standard. In one embodiment, the method detects cardiac tropinin levels as low as 5 fg/mL. As described herein, there are relevant diagnostic levels of cTn1 concentrations (<10-40 pg/mL) that can be detected in a clinically useful time frame. In particular, a sensitive assay allows for detection of a test subject that is not at risk of or having a cardiac event. In one embodiment, the levels of troponin in the test subject not at risk of acute coronary syndrome are less than about 300 fg/mL. In one embodiment, the complexes are detected with one or more second moieties that specifically bind cardiac troponin linked to a detectable molecule.

The methods described herein also allow for screening for cardiac cytotoxicity, e.g., in a non emergency room type setting. In one embodiment, the methods to detect cardiac troponin are employed to screen for the cardiac cytotoxicity of compounds administered to non human animals.

In one embodiment, a solid substrate comprises a plurality of different physically separated cardiac tropinin-specific binding moieties, e.g., cardiac troponin-specific antibodies that are specific for different epitopes of troponin are each present at different preselected positions on the solid substrate. Contacting the solid substrate with a physiological sample obtained from one or more time points can provide for a profile of the presence and/or amounts of those different epitopes at one or more times. Those profiles may be useful to differentiate between different acute coronary syndromes, prognosis, selection of therapies, or any combination thereof. Other factors which may be considered in the differential diagnosis, outcome or therapy selection include, but are not limited to, gender, ethnicity, age, smoking and/or diabetes. In one embodiment, where the solid substrate comprises an (first) antibody specific for one epitope of cardiac troponin, e.g., a monoclonal antibody, a polyclonal (second) antibody linked to the detectable molecule is employed to detect cardiac troponin bound to the first antibody. In one embodiment, where the solid substrate comprises a composition comprising (first) antibodies that bind more than one epitope of troponin, an antibody that binds to only one epitope of cardiac troponin is employed to detect cardiac troponin bound to the first antibodies.

Thus, the invention includes methods that employ an epitope-specific assay format to distinguish different forms of troponin and the changes of each form over time, so as to provide a differential diagnosis between UA, NSTEMI, ACS, AMI, and the like, at an earlier time point, which may reduce the need for additional cardiac diagnostic assays. Also included are methods for detecting troponin that are more sensitive, which employ a cutoff and/or slope measurements that may be used to differentiate one population, risk group or diagnosis from another for cardiac events.

Also provided is a computer-readable medium, with instructions thereon, which when executed by a processor of a computing device, cause the computing device to: receive first and second inputs indicative of detected amounts of complexes in first and second physiological fluid samples taken at respective first and second time points from a test subject; evaluate the first and second inputs as a function of one or more algorithms stored on the computer-readable medium to identify one or more cardiac conditions of the test subject; and provide an output indicative of the one or more identified cardiac conditions of the test subject.

Further provided is a system. The system includes a bus; a network interface coupled to the bus; a processor coupled to the bus; a memory coupled to the bus and holding an instruction set executable on the processor to: receive, over the network interface from a client, first and second inputs indicative of detected amounts of cardiac troponin concentrations of respective first and second physiological fluid samples taken at respective first and second time points from a test subject; evaluate the first and second inputs as a function of one or more algorithms held in the memory, the algorithms executable with regard to the first and second inputs to identify one or more cardiac conditions of the test subject; and provide, to the client over the network interface, an output indicative of the one or more identified cardiac conditions of the test subject.

### BRIEF DESCRIPTION OF THE FIGURES

FIG. 1A shows a dose response curves for cTnI concentrations after 30 minute (light gray triangles, T1), 1 hour (gray squares, T2), or 2 hour cTnI target incubation times (black diamonds, T3). The signal in the fully developed assay increases with the time allowed for the cTnI in the patient sample to come into contact with the capture antibody. The times to proceed from the T1 line to the T2 line to the T3 line depend upon the initial level of cTnI measured in the patient sample, and the assay parameters associated with the reagents in the cartridge (time, temperature, buffers, etc.). For example, antibody 817 (Nanogen) may be used as a capture antibody and PA1010 (Nanogen) may be used as the detection (probe) antibody.

FIG. 1B shows percent distribution (x-axis) versus measured cTnI concentration (y-axis) of UA, NSTEMI and normal patients determined at blood draw times of 0, 2 and 8 hours with the nanoparticle assay. Arrows high-light the progression of patient populations (UA for example—or NSTEMI) at each blood draw time point. Horizontal line at 2 pg/mL is the cut-off for the nanoparticle assay described herein (cut-off point is defined as the 99th %ile of reference population for the described assay, either the nanoparticle assay or a current commercial assay). Horizontal line at 10 pg/mL is the cut-off for an assay from Biomerieux. Other commercial assays (e.g., Siemens [Dade] Stratus CS, Abbott i-Stat POC) have cut-offs as high as (70-100 pg/mL). Additional ACS (acute coronary syndrome) patients diagnosed by the nanoparticle assay at each timed blood draw are within the dashed ellipse regions between the 99th percentile cut-offs of the commercial assays and the nanoparticle assay.

FIG. 1C shows percent distribution (x-axis) versus measured cTnI concentration (y-axis) of UA, NSTEMI and normal patients determined at blood draw times of 0, 2 and 8 hours with the nanoparticle assay. Arrows high-light the progression of patient populations (UA for example or NSTEMI) at each blood draw time point. Horizontal line at 2 pg/mL is the cut-off for the nanoparticle assay described herein (cut-off point is defined as the 99th %ile of reference population for the described assay, either the nanoparticle assay or a current commercial assay. Horizontal line at 10 pg/mL is the cut-off for an assay from Biomerieux. Other commercial assays (e.g., Siemens [Dade] Stratus CS, Abbott i-Stat POC) have cut-offs as high as (70-100 pg/mL). Early sensitivity (short assay times) in the nanoparticle assay is shown by the horizontal line at 2 pg/mL, while matured sensitivity (late assay times) is shown by the horizontal line at 500 fg/mL. Additional ACS (acute coronary syndrome) patients diagnosed within the dotted ellipse region at t=0 with aid of matured sensitivity.

FIG. 1D illustrates percent distribution (x-axis) versus measured cTnI concentration (y-axis) of UA, NSTEMI and normal patients determined at blood draw times of 0, 2 and 8 hours with the nanoparticle assay. Arrows high-light the progression of patient populations (UA for example or NSTEMI) at each blood draw time point. Horizontal line at 2 pg/mL is the cut-off for the nanoparticle assay described herein (cut-off point is defined as the 99th %ile of reference population for the described assay, either the nanoparticle assay or a commercial assay). Horizontal line at 10 pg/mL is the cut-off for an assay from Biomerieux. Other commercial assays (e.g., Siemens [Dade] Stratus CS, Abbott i-Stat POC) have cut-offs as high as (70-100 pg/mL). Additional patients diagnosed with ACS (acute coronary syndrome) and UA within the dotted ellipse at t=0 using a slope calculation to indicate rate of increase.

FIG. 2 illustrates differential measurement of cTnI epitopes using selected antibodies. Each column of the x-axis represents an anti-troponin Ab bound to the substrate (capture Ab). Each column of the y-axis represents a secondary anti-troponin Ab used to label the target bound to the antibody attached to the substrate. For each antibody, the clone name and troponin binding epitope are listed (e.g., clone 2B1.9 binds to troponin amino acid residues 41-53). The z-axis represents signal generated with the corresponding pair of antibodies.

FIG. 3 shows a hypothetical timeline for the generation cTnI epitopes resulting from a cardiac event where troponin is released into the circulation and antibody epitopes are exposed over time as the result of the degradation or metabolic processing by serum proteinases which alters the protein structure and exposes different regions of the troponin protein sequence to the bulk serum. An alternate, less sensitive, non-nanoparticle immunoassay based on detection of the sum of all epitopes rather than the appearance of individual epitopes is shown as a dotted line above the individual epitope graph. Less sensitive assays are unlikely to detect the appearance of individual epitopes and can not resolve the true 99th percentile cut-off for a normal population (which can only be observed with the a more sensitive assays such as the nanoparticle assay).

FIG. 4 illustrates a multi-time point, multi-metabolite diagnostic algorithm for analysis of the extent (amount) and time course of appearance of cardiac troponin epitopes.

FIG. 5 shows the distribution of cardiac troponin I levels in 181 normal patient samples collected from 18 to 30 year olds. The troponin values for each patient sample were generated using a standard curve of troponin I spiked into troponin free serum using a nanoparticle-based ultrasensitive troponin assay.

FIG. 6A illustrates the proportion of unstable angina patients with a detected positive cardiac troponin I (cTnI) at different time points using the current generation (cg) cTnI assay based upon a previously established cut-point (>0.10 ng/mL), and the lower limit of detection (≧0.04 ng/mL), and a nanoparticle (nano) based cTnI assay using the 99th percentile (0.008 ng/mL), the 10% CV (≧0.002 ng/mL), and the lower limit of detection (0.0005 ng/mL).

FIG. 6B shows the timing of detection of myocardial injury in patients with a non-ST elevation myocardial infarction using the current generation (cg) cTnI assay based upon a previously established cut-point (≧0.10 ng/mL), and the lower limit of detection (≧0.04 ng/mL), and a nanoparticle (nano) based cTnI assay using the 99th percentile (0.008 ng/mL), and the 10% CV (≧0.002 ng/mL).

FIG. 7A is a graph of mean signal intensity (y-axis) from the ultrasensitive rat troponin assay as a function of troponin target concentration (x-axis). A dose response in signal is observed from 10 to 2500 pg/mL troponin.

FIG. 7B is a table providing numerical data for each sample tested as part of the target titration. The percent CV was calculated using the six replicate spot measurements used to analyze the mean signal intensity from each assay. The recovered value was calculated based on a linear fit of the 0 to 400 fg/mL data demonstrating 10 fg/mL detection sensitivity.

FIG. 8 is a logical block diagram of a computing environment according to an example embodiment.

FIG. 9 is a block diagram of a computing device according to an example embodiment.

FIG. 10 is a block flow diagram of a computerized method according to an example embodiment.

FIG. 11 is a table showing patient results with a currently available commercial cTn assay.

FIG. 12 shows troponin measurements (pg/mL) using the ultrasensitive cTnI assay for 50 normal samples. Specific risk factors are listed, along with body mass index (BMI), ethnicity, gender, and age.

### DETAILED DESCRIPTION OF THE INVENTION

### Definitions

“Analyte” or “target analyte” is a substance to be detected in a test physiological sample using the present invention. The analyte can be any substance, e.g., a protein, or a set of related proteins, e.g., troponin isoforms and metabolites thereof.

“Capture moiety” is a specific binding member, capable of binding the analyte, which moiety may be in solution or directly or indirectly attached to a substrate. One example of a capture moiety includes an antibody bound to a support either through covalent attachment or by adsorption onto the support surface.

The term “ligand” refers to any organic compound for which a receptor or other binding molecule naturally exists or can be prepared. The term ligand also includes ligand analogs, which are modified ligands, usually an organic radical or analyte analog, usually of a molecular weight greater than 100, which can compete with the analogous ligand for a receptor, the modification providing means to join the ligand analog to another molecule. The ligand analog usually differs from the ligand by more than replacement of a hydrogen with a bond which links the ligand analog to another molecule, e.g., a label, but need not. The ligand analog can bind to the receptor in a manner similar to the ligand. The analog could be, for example, an antibody directed against the idiotype of an antibody to the ligand. For instance, a capture antibody may have a label that binds another molecule, e.g., the antibody is linked to biotin and strapetavidin is coated onto a substrate.

The term “receptor” or “antiligand” refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, avidin, protein A, barstar, complement component Clq, and the like. Avidin is intended to include egg white avidin and biotin binding proteins from other sources, such as streptavidin.

The term “antibody” refers to an immunoglobulin which specifically binds to and is thereby defined as complementary with a particular spatial and polar organization of another molecule, including recombinant antibodies such as chimeric antibodies and humanized antibodies. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. In addition, aggregates, polymers, and conjugates of immunoglobulins or their fragments can be used where appropriate so long as binding affinity for a particular molecule is maintained.

### Cardiac Troponin

In 2000, the American College of Cardiology and the European Society of Cardiology published a consensus document redefining myocardial infarction (J. Am. Coll. Cardiol., 36:2000)). The new definition combined rise and fall of biochemical markers of myocardial necrosis with any of the following conditions: ischemic symptoms, ECG changes, and coronary intervention. The recommended biochemical markers were the troponins (T or I). The changes stemmed largely from reports on the prognostic value of troponin indicating that troponin provided prognostic information incremental to previously available clinical factors (Braumwald et al., J. Am. Coll. Cardiol., 36:970 (2000); Antman et al., NEJM, 335:1342 (1996); Ohman et al. (NEJM, 335:1333 (1993)). In particular, troponin was more specific than CK/CK=MB for the diagnosis of myocardial infarction in the setting of associated skeletal muscle damage or injury including surgery. The troponins have higher sensitivity which allows for the detection of very small amounts of myocardial necrosis.

Traditional studies of the epidemiology of myocardial infarction have focused on infarction and have seldom reported on the clinical entity of acute coronary syndromes, with or without biomarker elevation. Part of the reason for this resides in the need for a standardized definition in epidemiology and the relative ease of standardizing the definition of myocardial infarction, contrasting with the more challenging task of defining acute coronary syndromes from an epidemiological biomarker elevation and with transient or absent electrocardiographic changes.

Acute coronary syndrome encompasses a spectrum of coronary artery diseases, including unstable angina, ST-elevation myocardial infarction (STEMI; often referred to as “Q-wave myocardial infarction”), and non-STEMI (NSTEMI; often referred to as “non-Q-wave myocardial infarction”). Differentiating acute coronary syndrome from noncardiac chest pain is a primary diagnostic challenge. Symptoms of acute coronary syndrome include chest pain, referred pain, nausea, vomiting, dyspnea, diaphoresis, and light-headedness. Some patients may present without chest pain. Pain may be referred to either arm, the jaw, the neck, the back, or even the abdomen. Pain radiating to the shoulder, left arm, or both arms somewhat increase the likelihood of acute coronary syndrome. Typical angina is described as pain that is substernal, occurs on exertion, and is relieved with rest. Atypical symptoms do not necessarily rule out acute coronary syndrome. However, a combination of atypical symptoms improves identification of low-risk patients.

Serum cardiac marker determinations play a vital role in the diagnosis of acute myocardial infarction. Characteristics of the most important serum cardiac markers for acute MI are, for CK, first positive at 3 to 8 hours and peaking at 12 to 24 hours, and in a serial assay has a 95% sensitivity and a 30% positive predictive value; for CK-MB, first positive at 4 to 6 hours, and peaking at 12 to 24 hours, and in a serial assay has 95% sensitivity and a 73% positive predictive value; assays for TnI and TnT, first positive at 4 to 10 hours and peaking at 8 to 28 hours, and for the peak times has a 89% sensitivity has a 72% positive predictive value while earlier times have a 35% sensitivity and 56% positive predictive value (Karras et al., Emerg. Med. Clin. North Am., 19:321 (2001)); Hamm et al., N. Engl. J. Med., 337:1648 (1997); Pope et al., Emerg. Med. Clin. North Am., 21:27 (2003); Balk et al., Ann. Emerg. Med., 37:478 (2001)).

Troponins (T, I, C) are found in striated and cardiac muscle. Because the cardiac and skeletal muscle isoforms of troponin T and I differ, they are known as the “cardiac troponins,” and are markers for the diagnosis of myocardial injury (Scirica et al., Semin. Vasc. Med., 3:363 (2003)). Troponin T and I generally have similar sensitivity and specificity for the detection of myocardial injury.

The cardiac troponins typically are measured at emergency department admission and repeated in six to 12 hours. The cardiac troponins may remain elevated up to two weeks after symptom onset, which makes them useful as late markers of recent acute myocardial infarction.

An elevated troponin T or I level is helpful in identifying patients at increased risk for death or the development of acute myocardial infarction (Karras et al., Emerg. Med. Clin. North Am., 19:321 (2001)). Increased risk is related quantitatively to the serum troponin level. The troponins also can help identify low-risk patients who may be home with close follow-up (Hamm et al., N. Engl. J. Med., 337:1648 (1997).

### Methods and Systems of the Invention

The invention provides sensitive methods and systems to assess cardiac events by detecting cTn levels and/or specific cTn isoforms. cTnI concentrations <10-40 pg/mL are undetectable in a clinically useful time frame using current commercially available cTnI assays. cTnI levels in the range of 10 fg/mL-10 pg/mL become measurable using the assays described herein, and the data described herein demonstrate that these ultrasensitive levels have relevance for diagnosing cardiac events. Further, by controlling the time of target incubation during the ultrasensitive assay, both rapid measurements in typical ranges for acute events (i.e., acute myocardial infarction which is measured currently at >10-40 pg/mL) as well as ultrasensitive measurements for earlier and differentiated diagnosis of other cardiac events (e.g., between NSTEMI, unstable angina, or earlier detection of AMI) may be achieved.

In one embodiment, the levels of cTn in a patient physiological sample, e.g., a physiological fluid sample, such as blood plasma, blood serum or saliva, or a tissue biopsy, e.g., are tested at two time points, for instance, using the same instrument. Due to the sensitivity of the present assay, a rapid test (a few minutes) measures levels (e.g., 10-100 pg/mL) currently requiring tens of minutes in state-of-the-art instruments. A positive reading for the rapid test likely obviates the need for further tests. If the first (rapid) test is negative, the second test may be conducted for a longer period of time (e.g., tens of minutes) to resolve cTn1 levels (e.g., <<10 pg/mL) not currently detectable by state-of-the-art instruments. In one embodiment, the slope of the change in cTn1 levels may be employed for a faster cardiac risk assessment or diagnosis of acute coronary syndrome (ACS) and/or a more differentiated diagnosis, e.g., between non-ST-elevation myocardial infarction (NSTEMI) and unstable angina (UA) when compared to the current practice of using a cutoff threshold to establish acute myocardial infarction. The change in cTn1 levels may be obtained using two successive blood draws or a more resolved assay (i.e., an assay where the sample is contacted with a capture moiety for a longer period of time) combined with a successive blood draw.

In one embodiment, one or more different types of capture moieties that bind to cardiac troponin may be immobilized onto the surface of a substrate, e.g., before contact with the sample. The capture moiety may be bound to the substrate by any conventional means including one or more linkages between the capture probe and the surface or by adsorption. In one embodiment, one or more different types of capture moieties that bind to cardiac troponin are contacted with the sample and in one embodiment, the resulting complex is immobilized onto the surface of a substrate. In another embodiment, the complex is not immobilized onto a substrate. The capture moiety and cardiac troponin may be specific binding pairs such as antibody-antigen or receptor-ligand. The presence of any target analyte-capture moiety complex is then detected, e.g., using probes having a detectable molecule. In one embodiment, where the detectable molecule is a nanoparticle, the presence of the nanoparticle may be detected by flow-based methods or detection may be enhanced by silver staining. Silver staining can be employed with any type of nanoparticle that catalyzes the reduction of silver. In one embodiment, the nanoparticles are made of noble metals (e.g., gold and silver). See Bassell et al., J. Cell Biol., 126:863 (1994); Braun-Howland et al., Biotechniques, 13:928 (1992). Silver staining has been found to provide a large increase in sensitivity for assays employing a single type of nanoparticle. For greater enhancement of the detectable change, one or more layers of nanoparticles may be used, each layer treated with silver stain as described in PCT/US01/21846.

In one embodiment, selection of various epitope- or modification-specific cTn antibodies, e.g., antibodies specific for, for instance, proteolytically degraded, phosphorylated, oxidized, or complexed cTn, allow for differential measurement of these various forms. In one embodiment, the assay combines various cTn antibodies to more accurately detect total cTn1 by detecting different forms simultaneously. In another embodiment, the assay includes simultaneous detection of each individual cTn1 form in a single test on a single sample based on multiple epitope detection. The results of such an assay may be useful to fine-tune the diagnosis or for additional diagnosis of cardiac events. Further, measuring various cTn forms via specific epitopes over time may allow for time-resolved differential diagnosis of changes in cTn1 profile, thereby permitting a more detailed diagnosis. Thus, the invention provides an assay that differentiates between acute coronary syndromes such as UA, NSTEMI, and/or acute myocardial infarction (AMI) based on the change in cTn levels over a relatively short time frame (e.g., over 5, 10, 15, 20, 25, 30 or more minutes, but less than 10 hours) or epitope-mediated troponin metabolite discrimination.

The results of the assays described herein allow earlier diagnosis of a patient upon initial presentation, and/or act as a substantially more sensitive baseline for further time points to measure changes in cTn1 levels or forms. Detection may employ a silver-amplified antibody probe array, a biobarcode assay, or a flow-based detection of nanoparticles (see, e.g., Nam et al., Science, 301:1884 (2003); Bao et al., Anal. Chem., 78:2055 (2006); U.S. Pat. Nos. 7,110,585; 6,506,564; 6,602,669; 6,645,721; 6,673,548; 6,677,122; 6,720,147; 6,730,269; 6,750,016; 6,767,702; 6,759,199; 6,812,334; 6,818,753; 6,903,207; 6,962,786; and 6,986,989, all of which are incorporated herein by reference). In these approaches, a solid substrate such as a microarray slide, magnetic bead, microwell plate or test tube is functionalized with different specific capture moieties (e.g., monoclonal antibodies) capable of specifically capturing the target or form of interest, e.g., at a defined epitope. A sample is allowed to contact the substrate for variable times which enables different levels of target detection. Once captured, a second set of nanoparticle-based detection probes functionalized with complementary moieties capable of specific and defined attachment to the captured target is introduced into the assay (note variations of this principle that are well established also can be used, including biotin-streptavidin interactions). Once this attachment is complete the signal of each unique capture moiety may be amplified by silver deposition on captured gold probe (array-based assay), unique reporter biobarcode oligos are released and detected on an array (biobarcode assay) or variable encoded probes are released and detected by laser-based flow. The assay results are read by a detection system (e.g., VerigeneID or a Tecan scanner) and an algorithm determines the quantity of each individual moiety and calculates the relative and total results over time. Additional samples taken over time are incorporated to determine and report any changes in quantity and slope of total and/or specific cTnI forms.

Thus, the present invention also relates to methods that utilize oligonucleotides as biochemical barcodes for detecting cardiac troponin in solution. The approach takes advantage of protein recognition elements functionalized with oligonucleotide hybridization events that result in the aggregation of gold nanoparticles which can significantly alter their physical properties (e.g. optical, electrical, mechanical). Each protein recognition element can be encoded with a different oligonucleotide and a physical signature associated with the nanoparticles that changes upon melting to decode a series of analytes in a multi-analyte assay, e.g., to detect specific epitopes of cardiac troponin.

In one embodiment, a particle complex probe is provided comprising a particle having oligonucleotides bound thereto, a DNA barcode, and a oligonucleotide having bound thereto a specific binding complement to a target analyte, wherein the DNA barcode has a sequence having at least two portions, at least some of the oligonucleotides attached to the particle have a sequence that is complementary to a first portion of a DNA barcode, the oligonucleotides having bound thereto a specific binding complement have a sequence that is complementary to a second portion of a DNA barcode, and wherein the DNA barcode is hybridized at least to some of the oligonucleotides attached to the particle and to the oligonucleotides having bound thereto the specific binding complement; contacting the sample with a particle complex probe under conditions effective to allow specific binding interactions between the analyte and the particle complex probe and to form an aggregated complex in the presence of analyte. Aggregate formation is then determined. In the presence of target analyte, aggregates are produced as a result of the binding interactions between the particle complex probe and the target analyte. The aggregates may be detected by any suitable means.

Each type of particle complex probe may contain a predetermined reporter oligonucleotide or barcode for a particular target analyte. In the presence of target analyte, nanoparticle aggregates are produced as a result of the binding interactions between the nanoparticle complex and the target analyte. These aggregates can be isolated and analyzed by any suitable means, e.g., thermal denaturation, to detect the presence of one or more different types of reporter oligonucleotides.

These aggregates may be isolated and subject to conditions effective to dehybridize the aggregate and to release the reporter oligonucleotide. The reporter oligonucleotide is then isolated. If desired, the reporter oligonucleotide may be amplified by any suitable means including PCR amplification. Analyte detection occurs indirectly by ascertaining for the presence of reporter oligonucleotide or biobarcode by any suitable means such as a DNA chip. After the DNA barcodes are isolated the presence of one or more DNA barcodes having different sequences are detected, wherein the identification of a particular DNA barcode is indicative of the presence of a specific target analyte in the sample.

### Exemplary Nanoparticles

Nanoparticles useful in the practice of the invention include metal (e.g, gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs. The size of the nanoparticles may be from about 5 nm to about 150 nm (mean diameter), e.g., from about 5 to about 50 nm, or from about 10 to about 30 nm. The nanoparticles may also be rods.

Methods of making metal, semiconductor and magnetic nanoparticles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, IEEE Transactions On Magnetics, 17:1247 (1981); Ahmadi et al., Science, 272:1924 (1996); Henglein et al., J. Phys. Chem., 99:14129 (1995); Curtis et al., Angew. Chem. Int. Ed. Engl., 27:1530 (1988).

Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32:41 (1993); Henglein, Top. Curr. Chem., 143:113 (1988); Henglein, Chem. Rev., 89:1861 (1989); Brus, Appl. Phys. A., 53:465 (1991); Bahncmann, in Photochemical Conversion and Storage of Solar Energy (eds. Pelizetti and Schiavello 1991), page 251; Wang and Herron, J. Phys. Chem., 95:525 (1991); Olshavsky et al., J. Am. Chem. Soc., 112:9438 (1990); Ushida et al., J. Phys. Chem., 95:5382 (1992).

Suitable nanoparticles are also commercially available from, e.g., Ted Pella, Inc. (gold), Amersham Corporation (gold) and Nanoprobes, Inc. (gold).

Gold colloidal particles have high extinction coefficients for the bands that give rise to their colors. The intense colors change with particle size, concentration, interparticle distance, and extent of aggregation and shape (geometry) of the aggregates, making these materials particularly attractive for colorimetric assays. Gold nanoparticles are stable and may readily be modified with thiol functionalities.

### Exemplary Solid Substrates

Any substrate which allows observation of the detectable change may be employed in the methods of the invention. Suitable substrates include transparent solid surfaces (e.g., glass, quartz, plastics and other polymers), opaque solid surface (e.g., white solid surfaces, such as TLC silica plates, filter paper, glass fiber filters, cellulose nitrate membranes, nylon membranes), and conducting solid surfaces (e.g., indium-tin-oxide (ITO), silicon dioxide (SiO₂), silicon oxide (SiO), silicon nitride, etc.)). The substrate can be any shape or thickness, but generally is flat and thin. In one embodiment, the substrates are transparent substrates such as glass (e.g., glass slides) or plastics (e.g., wells of microtiter plates).

In one embodiment, the present invention relates to the detection of metallic nanoparticles on a substrate. The substrate may have a plurality of spots containing specific binding moieties for one or more cardiac troponin related molecules (target analytes). One of the spots on the substrate may be a test spot (containing a test sample) for metallic nanoparticles complexed thereto in the presence of one or more target analytes. Another one of the spots may contain a control spot or second test spot. Further provided is a method for automatically detecting binding of moieties specific for cardiac troponin to at least some of the spots on the substrate. An image is acquired of the plurality of spots composed of metallic nanoparticles, with or without signal amplification, on the surface of the substrate. In one embodiment, the invention provides for methods for detection of gold colloid particles. In one embodiment, the nanoparticles are gold nanoparticles (either entirely composed of gold or at least a portion (such as the exterior shell) composed of gold) and amplified with silver or gold deposited post-hybridization on to the gold nanoparticles. In one embodiment, the metallic nanoparticles are subject to chemical signal amplification (such as silver amplification). Optionally, an optimal image is obtained based on an iterative process.

### Antibody Based Assays

Proteins such as cardiac troponin may be contacted with a panel of moieties such as aptamers or antibodies or fragments or derivatives thereof specific for the protein. The antibodies or other binding molecules may be affixed to a solid support such as a chip. Binding of proteins indicative of a particular epitope or isoform of cardiac troponin may be verified by binding to a detectably labelled secondary antibody or aptamer. For the labelling of antibodies, it is referred to Harlow and Lane, “Antibodies, A Laboratory Manual”, CSH Press, 1988, Cold Spring Harbor. For instance, antibodies against the proteins are immobilized on a solid substrate, e.g., glass slides or microtiter plates. The immobilized complexes can be labeled with a reagent specific for the protein(s). The reactants can include enzyme substrates, DNA, receptors, antigens or antibodies to provide, for example, a capture sandwich immunoassay.

Any of a variety of known immunoassay methods can be used for detection, including, but not limited to, immunoassay, using an antibody specific for the encoded polypeptide, immunoprecipitation, an enzyme immunoassay, e.g., by enzyme-linked immunosorbent assay (ELISA), radioimmunoassay (RIA), and the like.

Given that immunoassay sensitivity is defined not only by the detection system but by the binding affinities of the antibodies involved, it is possible for other detection methods used in commercially available technologies and also those previously defined in the academic literature but not commercially available to reach the assay sensitivities described in the present specification through the use of antibodies with particular binding affinities, or improvements to the detection method or assay methodology. Any of a variety of known immunoassay methods can be used for detection, including, but not limited to, immunoassay, using an antibody specific for the encoded polypeptide, e.g., by enzyme-linked immunosorbent assay (ELISA), radioimmunoassay (RIA), rolling circle amplification (RCA), immunoPCR (iPCR), magnetic bead based assays that utilize fluorescence and chemiluminescence, electrochemiluminescence and the like; and functional assays for the encoded polypeptide, e.g., binding activity or enzymatic activity.

As will be readily apparent to the ordinarily skilled artisan upon reading the present specification, the detection methods and other methods described herein can be varied. Such variations are within the intended scope of the invention. For example, in the above detection scheme, the probe for use in detection can be immobilized on a solid support, and the test sample contacted with the immobilized probe. Binding of the test sample to the probe can then be detected in a variety of ways, e.g., by detecting a detectable label bound to the test sample.

The methods generally include contacting the sample with an antibody specific for one or more forms of cardiac troponin, and detecting binding between the antibody and the one or more forms of cardiac troponin in the sample. The level of antibody binding indicates the susceptibility (at risk for, propensity or affirmative diagnosis) of the patient for a cardiac event. For example, where cardiac troponin levels are present at a level greater than that associated with a negative control level, the patient may be at risk of a cardiac event. Suitable controls include a sample known not to contain cardiac troponin; a sample contacted with an antibody not specific for cardiac troponin; a sample having a level of cardiac troponin associated with MI, UA or NSTEMI, or any combination thereof.

In one embodiment, the methods include contacting the sample with an antibody specific for the protein of interest (e.g., cTnI) and detecting binding between the antibody and molecules of the sample. The level of antibody binding (either qualitative or quantitative) indicates the susceptibility of the patient to a disease. For example, where the marker polypeptide is present at a level greater than that associated with a negative control level, then the patient is susceptive to disease.

In general, one of the binding moieties, e.g., antibody, is detectably labeled, either directly or indirectly. Direct labels include radioisotopes; enzymes having detectable products (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like. Indirect labels include second antibodies specific for antibodies specific for cardiac troponin (“first specific antibody”), wherein the second antibody is labeled as described above; and members of specific binding pairs, e.g., biotin-avidin, and the like.

One of the binding moities, e.g., antibody, may be attached (coupled) to an insoluble support, such as a polystyrene plate or a bead. In one embodiment, the sample may be brought into contact with the immobilized antibody and the support washed with suitable buffers followed by contact with a detectably labeled specific antibody. In one embodiment, the sample may be brought into contact with and immobilized on a solid support or carrier, such as nitrocellulose, that is capable of immobilizing soluble proteins. The support may then be washed with suitable buffers followed by contacting with an optionally detectably labeled first specific antibody. Detection methods are known in the art and are chosen as appropriate to the signal emitted by the detectable label. Detection is generally accomplished in comparison to suitable controls, and to appropriate standards.

In one embodiment, the antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls, and to appropriate standards.

Polypeptide arrays provide a high throughput technique that can assay a large number of polypeptides in a sample. This technology can be used as a tool to test for presence of a marker polypeptide and assessment of cardiac disease. Of particular interest are arrays which comprise a probe for detection of one or more of the marker polypeptides of interest.

A variety of methods of producing arrays of binding molecules, as well as variations of these methods, are known in the art and contemplated for use in the invention. For example, arrays can be created by spotting binding moieties onto a substrate (e.g., glass, nitrocellulose, and the like) in a two-dimensional matrix or array having bound probes. Arrays also can be created by spotting polypeptide probes onto a substrate in a three-dimensional matrix (e.g. hydrogel) or array having bound probes. The probes can be bound to the substrate by either covalent bonds or by non-specific interactions, such as hydrophobic interactions.

Samples of cardiac troponin can be detectably labeled (e.g., using radioactive or fluorescent labels) and then contacted with the binding moieties. Alternatively, the test sample can be immobilized on the array, and the binding moieties detectably labeled and then applied to the immobilized polypeptides. In one embodiment, a binding moiety is detectably labeled. In other embodiments, the binding moiety is immobilized on the array and not detectably labeled. In such embodiments, the sample is applied to the array and bound molecules are detected using labeled binding moieties. In one embodiment, the secondary label probes can be introduced in a direct sandwich format where a primary antibody is bound to the substrate, and the secondary antibody is directly attached to the label such as a gold nanoparticle, which “sandwiches” the target protein when both the primary and secondary antibody binds to epitopes of the target. An alternative methodology well known in the art is to use a secondary antibody in an indirect sandwich assay where the antibody is label with a hapten such as biotin, which can then recognize a streptavidin or avidin molecule which is directly labeled or indirectly labeled.

Other methods well known in the art are competitive immunoassay formats where the signal the presence of known amount of target added to the sample competes against an unknown amount of target present in the sample.

Examples of such protein arrays are described in the following patents or published patent applications: U.S. Pat. No. 6,225,047; PCT International Publication No. WO 99/51773; U.S. Pat. No. 6,329,209; PCT International Publication No. WO 00/56934; and U.S. Pat. No. 5,242,828.

### Exemplary Assays

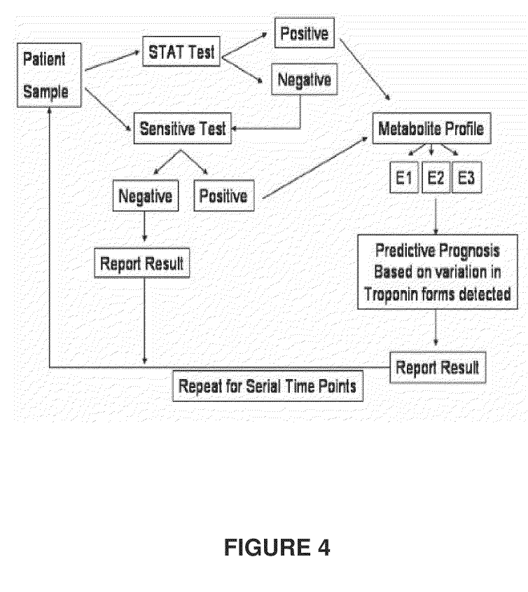

In one embodiment, a solid substrate such as a glass or plastic slide, e.g., a microarray slide, magnetic bead, microwell plate or test tube is functionalized with different specific capture moieties (e.g., monoclonal or polyclonal antibodies) capable of specifically isolating the cTn target or form of interest, e.g., using an antibody that binds a defined epitope. A physiological sample is contacted with the functionalized substrate for one or more periods of time. Different incubation times allow for different levels of target detection. Once captured, one or more nanoparticle-based detection probes functionalized with moieties capable of specific and defined binding to the captured target are added. Then the signal of each unique capture moiety is detected by, for instance, silver deposition on captured gold containing moieties (array-based assay), the release and detection of unique reporter biobarcode oligonucleotides on an array (biobarcode assay) or the release and detection of variable encoded probes by laser-based flow techniques. The assay results are read by a detection system (e.g., VerigeneID). An algorithm (FIG. 4) may be employed to determine the quantity of total cTn or of each individual moiety and calculate the relative and total results over time. Additional samples may be taken later in time and the results of those additional samples compared to earlier results to determine and report any changes in quantity and/or slope of total and/or specific cTn1 forms.

The signal in the assay increases with the time allowed for the cTn1 in the patient sample to come into contact with the capture antibody on the solid substrate, e.g., the Verigene cartridge. The time required to proceed from T1, T2 to T3 line depends upon the initial level of cTn1 in the patient sample and assay reagents and conditions in the cartridge (time, temperature, buffer, and the like).

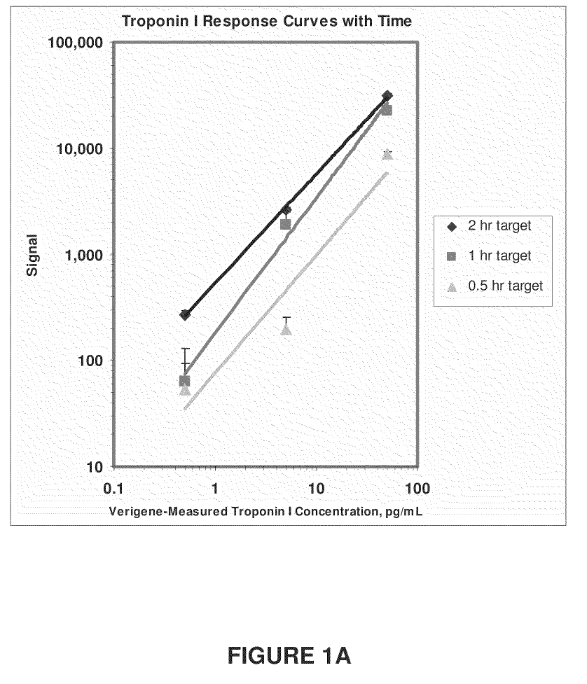

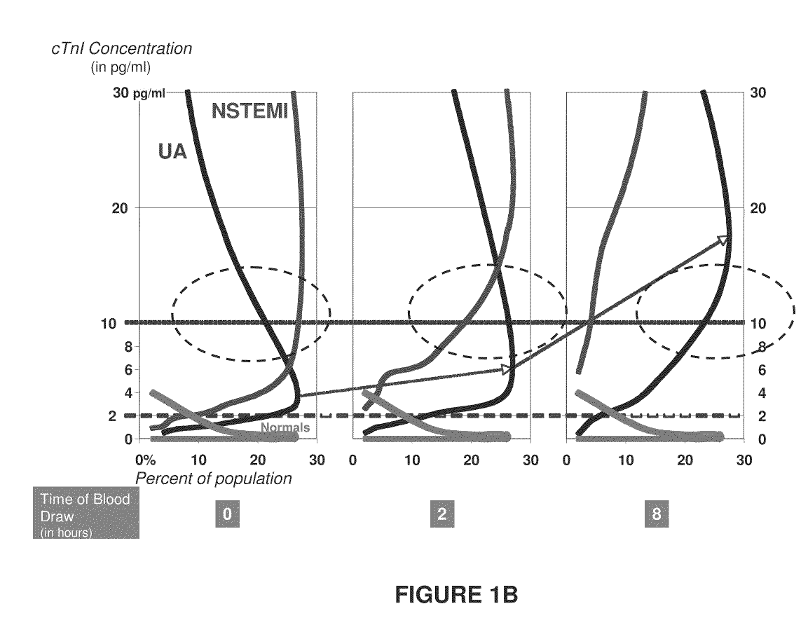

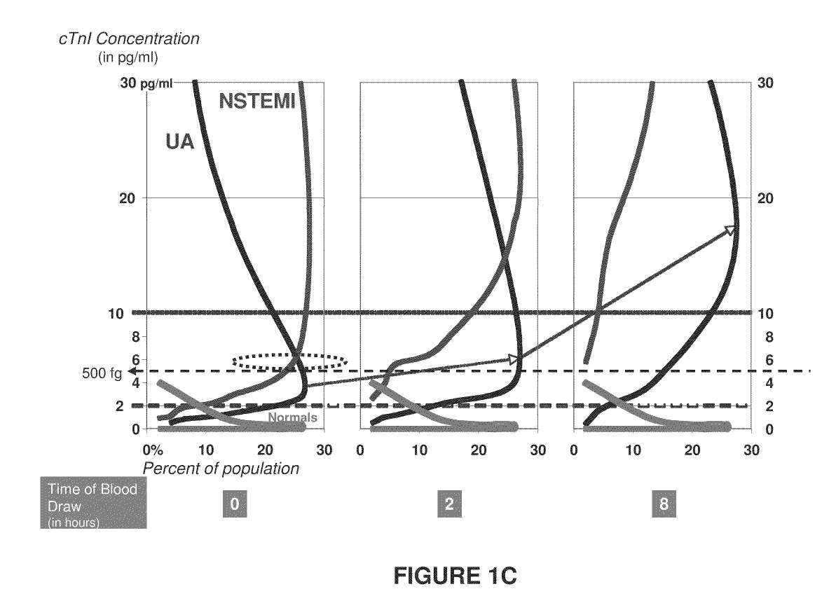

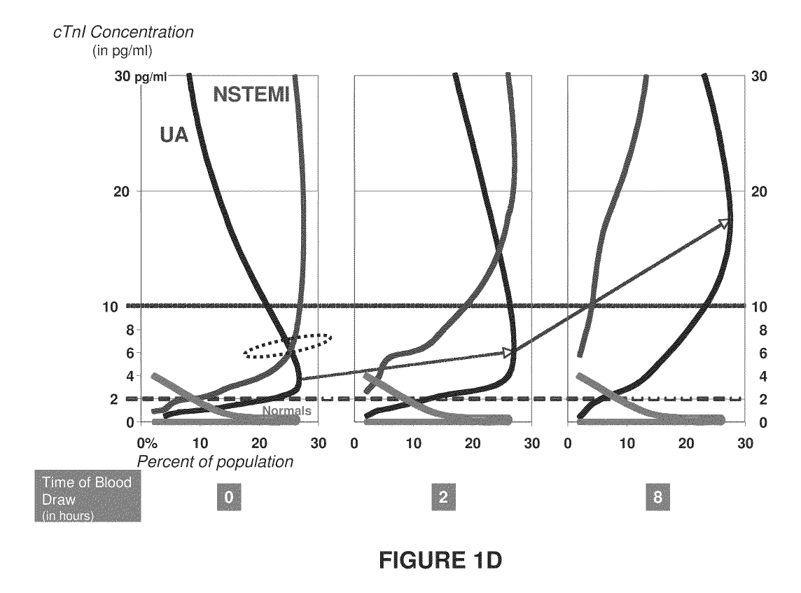

For example, as patient presents with chest pain. From an initial blood draw, three sample measurements of cTn1 are conducted. Separate cTn1 specific solid substrates with the sample are incubated for T1, T2 and T3 minutes. A signal at T1 on the light grey triangle in FIG. 1A response line progresses to a signal of T3 on the dark grey square response line. A quick test can be used to rule in AMI, as low pg/mL sensitivity using assays described herein can be obtained with as little as 10 minute target incubation. Samples from patients that are negative on the quick test can be measured at 30 minutes, which is demonstrating sensitivities in the hundreds of fg/mL, enough to measure ACS, UA, and the like, all from one blood draw at time zero. Disease progression can be measured by a subsequent patient sample obtained at a later time and assayed in the same fashion.

The higher sensitivity of the current assay, such as one employing silver deposition allows ruling in of NSTEMI significantly earlier and ruling in of UA significantly earlier for the small portion that may be identified using current assays a larger portion which would not otherwise be identified with current assays, and in a differentiated fashion from NSTEMIs for time points later than that needed for 100% NSTEMI sensitivity (e.g., 2 hours) the assay also allows ruling out normals significantly earlier by achieving near 100% sensitivity for ACS not otherwise possible.

In addition, the assay may provide for the ability to distinguish between NSTEMI, UA, ACS, and AMI, based on the different forms of troponin that are present in the sample.

Using changes in values of serial blood draws provided by the higher-sensitivity plus increasing sensitivity for each individual blood draw using “sensitivity maturation,” the use of the current assay provides ruling in of NSTEMI significantly earlier and ruling in of UA significantly earlier for the small portion that may be identified using current assays a larger portion which would not otherwise be identified with current assays, and in a differentiated fashion from NSTEMIs for time points later than that needed for 100% NSTEMI sensitivity (e.g., 2 hours) ruling in of UA and in a differentiated fashion from NSTEMIs using two time points (e.g., 2 hours); and ruling out normals significantly earlier by achieving near 100% sensitivity for ACS, not otherwise possible, using not only values but the slopes of concentrations (FIG. 1C).

Using changes in values of serial blood draws provided by the higher-sensitivity plus increasing sensitivity for each individual blood draw even without “sensitivity maturation,” the use of the current assay ruling in of NSTEMI significantly earlier and ruling in of UA significantly earlier for the small portion that may be identified using current assays a larger portion which would not otherwise be identified with current assays, and in a differentiated fashion from NSTEMIs for time points later than that needed for 100% NSTEMI sensitivity (e.g., 2 hours); and ruling out normals significantly earlier by achieving near 100% sensitivity for ACS, not otherwise possible, using not only values but the slopes of concentrations (FIG. 1D).

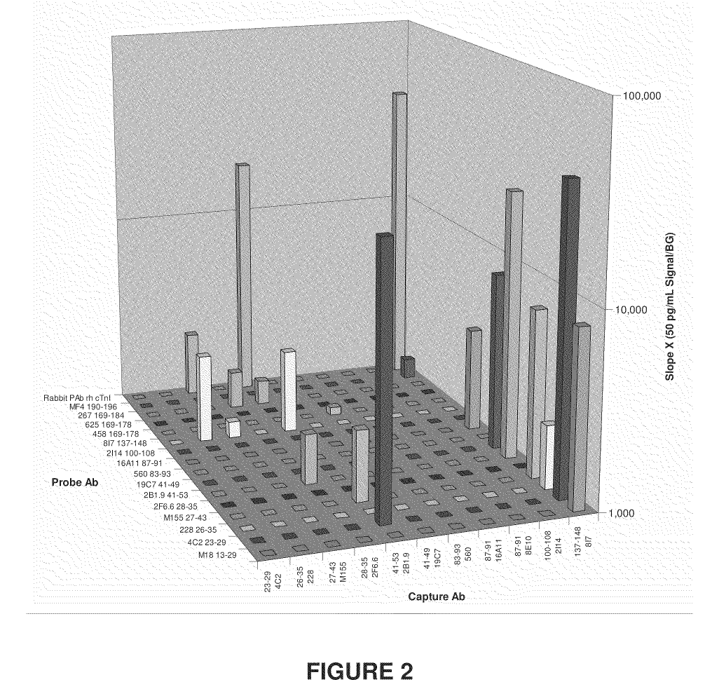

FIG. 2 shows results for differential measurement of cTn1 epitopes using selected specific antibodies. A cTn1 sample is introduced to an array of antibodies with known specificities for various parts of the cTn1 molecule, or different forms of the cTn1 molecule. After capture of the various cTn1 molecules, a probe or probes functionalized with antibody or antibodies to complementary parts of the molecules of interest are introduced. Capture of the probe(s) and the signal generated depends on the absence, presence and/or quantity of the cTn1 molecules, parts or modifications of interest. These results can be correlated with cardiac events to allow for more specific diagnosis.

The dotted line indicates accumulation of total cTn 1 products over time. Straight lines indicate accumulation of various cTn1 forms over time. Curved lines indicate change in distribution of various cTn1 forms over time. Squiggle lines indicate present limits of detection for standard assays. E1, E2, E3 correlate with various disease states, allowing more complete diagnoses.

A patient sample is split and added to each of two solid supports, for instance, cartridges in Verigene System (see U.S. Pat. No. 7,110,585, which is incorporated herein by reference). The first test result is determined in a few minutes. If negative, sensitive test results determined after longer incubation are needed. Total results and individual forms of troponin (Metabolite Profile) are also reported. Repeat for additional time points to measure changes in troponin forms over time.

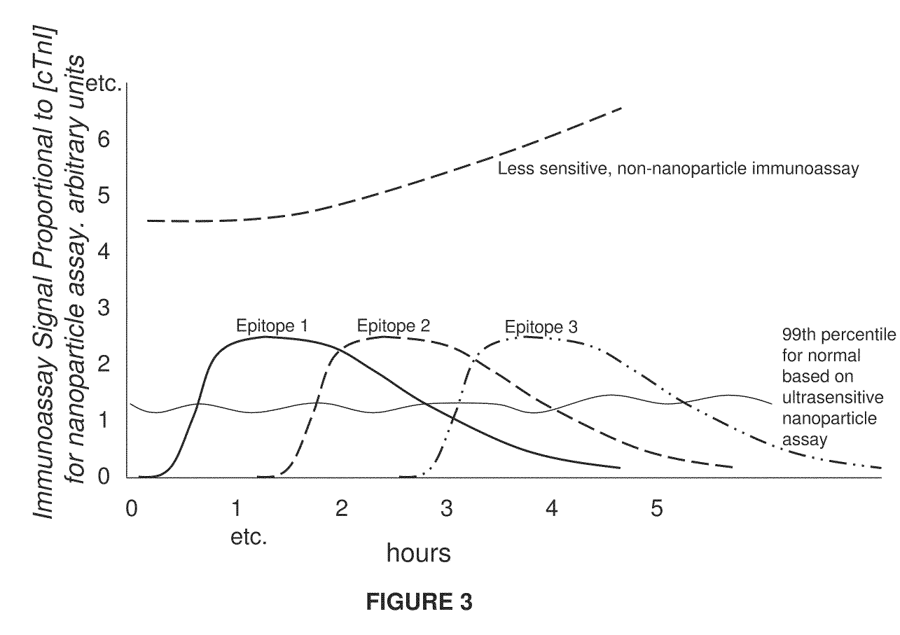

By employing an epitope-specific assay format and algorithm (FIG. 4) capable of distinguishing between different forms of troponin (FIG. 2) and the changes of each form over time (FIG. 3), a differential diagnosis can be made between UA, NSTEMI, ACS, and AMI (FIGS. 1B-E) sooner (FIG. 1A) and more completely than other assays, as well as reducing the need for additional cardiac diagnostic assays.

### Algorithms and Computer Applications

The invention also provides a variety of computer-related embodiments. Specifically, the automated means for performing the methods described above may be controlled using computer-readable instructions, i.e., programming. Accordingly, in some embodiments the invention provides computer programming for analyzing and comparing protein patterns present in a sample, wherein the comparing indicates the presence or absence of a disease.

In another embodiment, the invention provides computer programming for analyzing and comparing a first and second protein patterns from samples taken from a subject in at least two different time points, wherein the first pattern is indicative of a disease. In such embodiments, the comparing provides for monitoring of the progression of the disease from the first time point to the second time point.

The methods and systems described herein can be implemented in numerous ways. In one embodiment of particular interest, the methods involve use of a communications infrastructure, for example the internet. Several embodiments of the invention are discussed below. It is also to be understood that the present invention may be implemented in various forms of hardware, software, firmware, processors, or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

For example, during or after data input by the user, portions of the data processing can be performed in the user-side computing environment. For example, the user-side computing environment can be programmed to provide for defined test codes to denote platform, carrier/diagnostic test, or both; processing of data using defined flags, and/or generation of flag configurations, where the responses are transmitted as processed or partially processed responses to the reviewer's computing environment in the form of test code and flag configurations for subsequent execution of one or more algorithms to provide a results and/or generate a report in the reviewer's computing environment.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which generally includes test data (e.g., protein levels or patterns tested), and test result data (e.g., the levels of specific proteins within a sample). This information received can be stored at least temporarily in a database, and data analyzed in comparison to a library of known protein patterns to be indicative of the presence or absence of a disease.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In an embodiment of particular interest, all or a portion of the input data and/or all or a portion of the output data (e.g., usually at least the protein levels known to be indicative of the presence or absence of a disease) are maintained on a server for access, preferably confidential access. The results may be accessed or sent to professionals as desired.

A system for use in the methods described herein generally includes at least one computer processor (e.g., where the method is carried out in its entirety at a single site) or at least two networked computer processors (e.g., where protein pattern data for a sample obtained from a subject is to be input by a user (e.g., a technician or someone performing the activity assays)) and transmitted to a remote site to a second computer processor for analysis (e.g., where the protein pattern data is compared to a library of protein patterns known to be indicative of the presence or absence of a cardiac disease), where the first and second computer processors are connected by a network, e.g., via an intranet or internet). The system can also include a user component(s) for input; and a reviewer component(s) for review of data, and generation of reports, including detection of disease, differential diagnosis or monitoring the progression of a disease. Additional components of the system can include a server component(s); and a database(s) for storing data (e.g., as in a database of report elements, e.g., a library of protein patterns known to be indicative of the presence or absence of a disease, or a relational database (RDB) which can include data input by the user and data output. The computer processors can be processors that are typically found in personal desktop computers (e.g., IBM, Dell, Macintosh), portable computers, mainframes, minicomputers, or other computing devices.

The networked client/server architecture can be selected as desired, and can be, for example, a classic two or three tier client server model. A relational database management system (RDMS) either as part of an application server component or as a separate component (RDB machine) provides the interface to the database.

In one embodiment, the architecture is provided as a database-centric user/server architecture, in which the user application generally requests services from the application server which makes requests to the database (or the database server) to populate the activity assay report with the various report elements as required, especially the assay results for each activity assay. The server(s) (e.g., either as part of the application server machine or a separate RDB/relational database machine) responds to the user's requests.

The input components can be complete, stand-alone personal computers offering a full range of power and features to run applications. The user component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe and offers data management, information sharing between clients, network administration and security. The application and any databases used can be on the same or different servers.

Other computing arrangements for the user and server(s), including processing on a single machine such as a mainframe, a collection of machines, or other suitable configuration are contemplated. In general, the user and server machines work together to accomplish the processing of the present invention.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

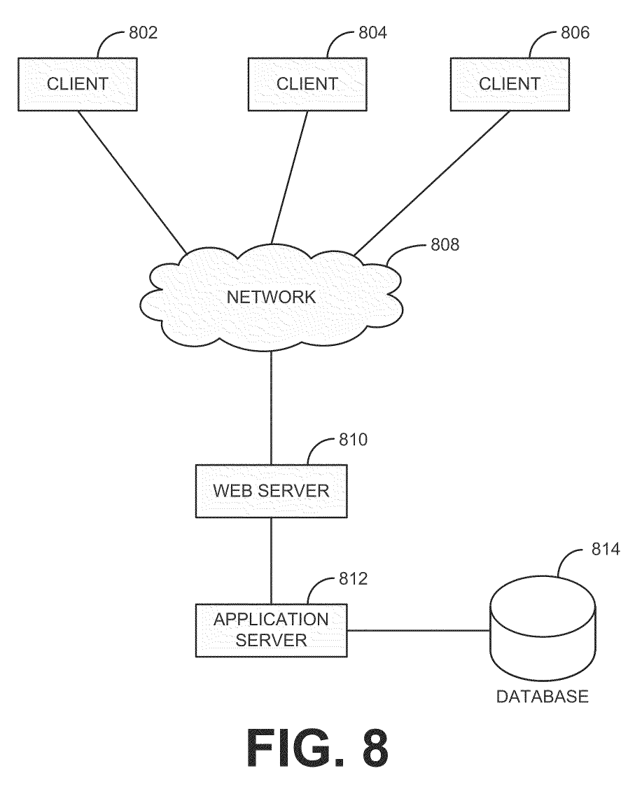

FIG. 8 is a logical block diagram of a computing environment 800 according to an example embodiment. The computing environment 800 includes an application server 812 accessible by client computing devices 802, 804, 806 over a network 808 via a web server 814. The application server 812 provides services to requesting a requesting client 802, 804, 806. Services may be requested over the network 808 via web pages, applications, or processes of the client 802, 804, 806.

For example, the application server 812 may receive a request from the web server 810 that originated with one of the clients 802, 804, 806. The request may be a request to detect an acute coronary syndrome as a function of two input values which may be provided with the request. The service of the application server may be operable to receive first and second inputs indicative of detected amounts of cardiac troponin concentrations which may include cardiac troponin concentrations less than 10 pg/mL. The service may then evaluate the first and second inputs as a function of one or more algorithms to identify one or more cardiac conditions of the particular test subject, such as a test subject. The service may then provide an output indicative of the one or more identified cardiac conditions.

In some embodiments, the computing environment 800 may also include a database 814. The service of the application server 812 may access data from and store data to the database 814. For example, the one or more algorithms to identify one or more cardiac conditions of the particular test subject may be retrieved from the database 814. In some embodiments, the input received in a client request and an output provided in response to a request may also be stored in the database 814.

The stored algorithms, in various embodiments, may include algorithms encoded in computer executable code, such as a computer program or module, to perform one or more of the methods described herein. Such algorithms may detect cardiac conditions as a function of the two inputs, or one or more than two inputs, to detect such coronary positions as acute coronary syndrome, unstable angina, non-ST elevation myocardial infarction, a normal cardiac condition.

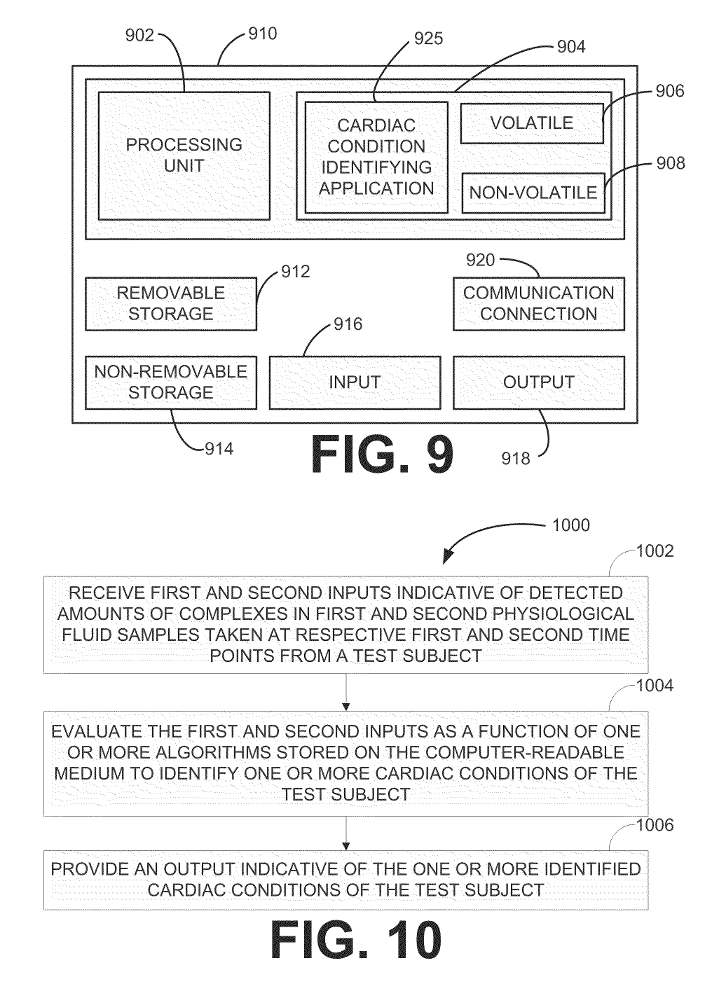

FIG. 9 is a block diagram of a computing device according to an example embodiment. In one embodiment, multiple such computer systems are utilized in a distributed network to implement multiple components in a transaction based environment. An object oriented, service oriented, monolithic, or other architecture may be used to implement such functions and communicate between the multiple systems and components. One example computing device in the form of a computer 910, may include a processing unit 902, memory 904, removable storage 912, and non-removable storage 914. Memory 904 may include volatile memory 906 and non-volatile memory 908. Computer 910 may include—or have access to a computing environment that includes—a variety of computer-readable media, such as volatile memory 906 and non-volatile memory 908, removable storage 912 and non-removable storage 914. Computer storage includes random access memory (RAM), read only memory (ROM), erasable programmable read-only memory (EPROM) & electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions. Computer 910 may include or have access to a computing environment that includes input 916, output 918, and a communication connection 920. The computer may operate in a networked environment using a communication connection to connect to one or more remote computers, such as database servers. The remote computer may include a personal computer (PC), server, router, network PC, a peer device or other common network node, or the like. The communication connection may include a Local Area Network (LAN), a Wide Area Network (WAN) or other networks.

Computer-readable instructions stored on a computer-readable medium are executable by the processing unit 902 of the computer 910. A hard drive, CD-ROM, and RAM are some examples of articles including a computer-readable medium. For example, a computer program 925 capable of implementing one or more of the methods described herein to identify various cardiac conditions, identify a condition as a function of various protein patterns, and other methods. An example of one such method is illustrated and described with regard to FIG. 10.

FIG. 10 is a block flow diagram of a computerized method 1000 according to an example embodiment. The computerized method 1000 is a method that may be performed by a computer application executing on a stand alone computer, on a server accessible to client computing devices, or in a number of other device and networked computing environments. The method 1000 is an example method that may be performed to identify a cardiac condition of a test subject, such as a test subject, based on results of a number of tests. For example, the method 1000 may include receiving 1002 first and second inputs indicative of detected amounts of complexes in first and second physiological fluid samples taken at respective first and second time points from a test subject. The computerized method 1000 further includes evaluating 1004 the first and second inputs as a function of one or more algorithms to identify one or more cardiac conditions of the test subject. An output indicative of the one or more identified cardiac conditions of the test subject may then be provided 1006. The output may be provided 1006 as a displayed output on a monitor or other display device. In other embodiments, the output may be provided as a data value from a process executing on a server. In some embodiments of the method 1000 the amount of complexes are detected and received from a device that detects the complex amounts in the first and second physiological fluid samples by forming the complexes by contacting the physiological fluid samples and a solid substrate having one or more cardiac-troponin specific antibodies.

The invention will be further described by the following nonlimiting examples.

### Example 1

### cTnI Assay Sensitivity as a Function of Time

In the example shown in FIG. 1A, known amounts of cTnI (50, 5, 0.5 and 0 pg/mL of cTnI NIST standard spiked into troponin depleted serum) were introduced to an array of anti-troponin antibodies (81-7 antibody analyzed) deposited on Codelink substrates (SurModics, Eden Prairie, Minn.) in separate assays. For each cTnI concentration, three different incubation times were tested (0.5 hours (hr), 1 hr, and 2 hrs). After capture of the cTnI molecules, a x-cTnI probe comprised of 13 nm diameter gold particle functionalized with 19C7 anti-troponin antibodies was added to each sample. Detection of the gold particle labeled troponin was achieved by catalytically reducing silver onto the surface of the gold particles followed by imaging light scattered by the silver amplified gold particles using a surface scanner such as a Tecan LS (Tecan USA) or a Verigene ID (Nanosphere, Northbrook, Ill.) detection system. These data demonstrate that different levels of sensitivity are achieved by incubating the sample containing cTnI target for different periods of time.

### Example 2

### Measurements on Patient Samples

### Methods

The nanoparticle-based assay quantifies an optical signal resulting from selective detection of the cTn1 molecule through analyte-specific capture of gold nanoparticle probes, followed by subsequent signal enhancement. Briefly, capture antibody (mouse monoclonal, Nanogen) was contact-spotted in triplicate onto CodeLink activated microarray slides along with controls in ten sub-arrays and assembled into a hybridization chamber. Fifty microliters of sample was incubated for 90 minutes at 35° C. Wells were washed with 0.3% Tween 20 in phosphate-buffered saline (PBS) pH 7.4, then incubated with detection antibody (rabbit polyclonal, Nanogen) in 1% BSA, 0.3% Tween in PBS pH 7.4 and incubated for 20 minutes. Wells were washed again, then incubated in 150 pM gold nanoparticles for 10 minutes. Slides were washed, followed by a 150 mM sodium nitrate, pH 7.5 wash. Capture substrates were immersed in signal enhancement reaction mix for 9 minutes at 19° C., and rinsed. Slides were dried and scanned (see Example 1).

### Results

Three sample measurements of cTnI were started from an initial blood draw using the described ultrasensitive cTnI assay. An example of separate cTnI assays performed for T1 (0.5 hours), T2 (1 hour) and T3 (2 hours) is shown in FIG. 1A. A signal at T1 on the light grey triangle response line progresses to a signal of T3 on the dark grey square response line. A quick test (10 minute target incubation) can be used to rule in AMI (data not shown). Patients that are negative on the quick test can be measured on the 30 minute target incubation test (T1), which has sensitivities in the hundreds of fg/mL, enough to measure ACS, UA, etc., all from one blood draw at time zero. Disease progression can be measured by a subsequent patient sample obtained at a later time (e.g., T2 or T3) and assayed in the same fashion.

As shown in FIG. 1B, the higher sensitivity of the nanoparticle-based assay enables ruling in of NSTEMI significantly earlier; ruling in of UA: i. significantly earlier for the small portion that may be identified using current assays, ii. an additional larger portion which would not otherwise be identified with current assays, iii. in a differentiated fashion from NSTEMIs for time points later than that needed for 100% NSTEMI sensitivity (e.g., 2 hours); and ruling out normals significantly earlier by achieving near 100% sensitivity for ACS not otherwise possible.

FIG. 1C illustrates changes in troponin values from serial blood draws which were detected earlier by the higher-sensitivity assay described herein, including the concept of “sensitivity maturation” which increases sensitivity for each individual blood draw by using a longer incubation time as described above. The use of this assay to measure cTnI as shown allows for ruling in of NSTEMI significantly earlier; and ruling in of UA significantly earlier for the small portion that may be identified using current assays, to rule in an additional larger portion which would not otherwise be identified with current assays, in a differentiated fashion from NSTEMIs using two time points (e.g., 2 hours), and ruling out normals significantly earlier by achieving near 100% sensitivity for ACS, not otherwise possible, using not only values but the slopes of concentrations. In addition, the assay may distinguish between NSTEMI, UA, ACS, AMI, etc. based on the different forms of troponin that are present in the sample.

In FIGS. 1B-D, the distributions of patient population for each disease type are shown to change between time points. The lower cutoffs (shown by dotted horizontal lines in FIGS. 1B-D) can be used for earlier risk assessment or diagnosis of patients using the ultrasensitive assay. Additionally, slopes are shown by arrows from one time point to another for the same individual or population in FIGS. 1C and 1D. The slope can be used for earlier risk assessment or diagnosis of patients using the ultrasensitive assay. The cutoff and slope measurements also can be used to differentiate one population, risk group or diagnosis from another.

### Example 3

### Differential Measurement of cTnI Epitopes Using Selected Specific Antibodies and Diagnostic Algorithms

In this example, a cTnI sample (50 pg/mL of cTnI NIST standard spiked into troponin depleted serum) was introduced to an array of anti-troponin antibodies deposited on Codelink substrates (labeled Capture Ab), FIG. 2. The capture antibodies have known specificities for various parts of the cTnI molecule, or different forms of the cTnI molecule, FIG. 2. After capture of the various cTnI molecules, a probe or probes functionalized with antibody to complementary parts of the molecules of interest were introduced. In this example, the secondary antibodies were attached to 13 nm diameter gold particles and introduced in separate assays. Each of the anti-troponin secondary antibodies (labeled probe Ab) binds to a unique epitope of the cTnI molecule. Capture of the probe(s) and the signal generated depends on the absence, presence and/or quantity of the cTnI molecules, parts or modifications of interest. This experiment demonstrates that different combinations of antibodies uniquely detect this specific form of cTnI.

These results can be correlated with important cardiac events to allow for more specific diagnosis. By combining different antibodies that uniquely identify different forms of troponin, specific algorithms can be developed for diagnosing cardiac events. A hypothetical timeline for the generation of cTnI epitopes resulting from a cardiac event where troponin is released into circulation and antibody epitopes are exposed over time as the result of the degradation or metabolic processing by serum proteinases which alters the protein structure and exposes different regions of troponin protein sequence to the bulk serum is shown in FIG. 3. An alternate, less sensitive, non-nanoparticle immunoassay based on detection of the sum of all epitopes rather than the appearance of individual epitopes is shown above as a dotted line. The less sensitive assay can not detect the appearance of individual epitopes and can not resolve the true 99th percentile cut-off for a normal population (which can only be observed with the more sensitive nanoparticle assay). Referring to FIGS. 1B-D for relative changes in specific cardiac diagnoses, Epitope 1, Epitope 2, Epitope 3 in FIG. 3 would correlate with various disease states shown in FIG. 1, allowing more complete diagnoses.

FIG. 4 further illustrates a multi-timepoint, multi-metabolite diagnostic algorithm which may be used in conjunction with the epitope specific assay format for predictive diagnosis of cardiac events. The patient sample is split into two assays which are performed for different incubation periods (Stat test and sensitive test in FIG. 4). For example, the two assays can be performed in two cartridges on the Verigene system (see U.S. Pat. No. 7,110,585, which is incorporated herein by reference). Stat test results are determined in a few minutes. If negative, refer to sensitive test results determined after longer incubation. Total results and individual forms of troponin (Metabolite Profile) are reported. Repeat for additional time points to measure changes in troponin forms over time.

### Example 4

### Measurement of Normal Range Using the Nanoparticle cTnI Assay

Cardiac troponin (cTn) detects myocardial necrosis and defines myocardial infarction (MI) when concentrations are increased above the 99th percentile in patients with symptoms of ischemia (Thygesen et al., Europ. Heart J., 28:2525 (2007)). The current generation of cTn assays has demonstrated 99^{th} percentiles ranging from 0.025 to 0.06 μg/L with lower limits of detection to 0.006 μg/L (Jaffe et al., J. Am. Coll. Cardiol., 48:1 (2006); Apple et al., Clin. Chem., 53:1558 (2007)). Jaffe expounds on the need for high sensitive cTn assays that can measure small increases in cTn across the first couple of hours after clinical presentation; distinguishing between patients with acute and chronic heart disease: predicated on the observation that the true 99th percentile is 10-100 fold below what current commercial assays measure (Jaffe, J. Am. Coll. Cardiol., 48:1763 (2006)). The goal of this study was to evaluate the performance of the ultrasensitive nanoparticle-based cTnI assay described herein.

### Methods

The nanoparticle-based assay quantifies an optical signal resulting from selective detection of the cTnI molecule through analyte-specific capture of gold nanoparticle probes, followed by subsequent signal enhancement. Briefly, capture antibody (mouse monoclonal, Nanogen) was contact-spotted in triplicate onto CodeLink activated microarray slides along with controls in ten sub-arrays and assembled into a hybridization chamber. Fifty microliters of sample was incubated for 90 minutes at 35° C. Wells were washed with 0.3% Tween 20 in phosphate-buffered saline (PBS) pH 7.4, then incubated with detection antibody (rabbit polyclonal, Nanogen) in 1% BSA, 0.3% Tween in PBS pH 7.4 and incubated for 20 minutes. Wells were washed again, then incubated in 150 pM gold nanoparticles for 10 minutes. Slides were washed, followed by a 150 mM sodium nitrate, pH 7.5 wash. Capture substrates were immersed in signal enhancement reaction mix for 9 minutes at 19° C., and rinsed. Slides were dried and scanned (see Example 1).

### Results

A standard curve was made with serial dilutions (n=8 replicates) using human cTn complex NIST SRM 2921 in cTn-depleted serum. Linearity curves were made by serial dilution of the NIST standard in pooled cTn-negative serum; and also assayed for cTnI on the Abbott Architect, Siemens Stratus CS, Siemens Ultra, and Beckman Access. Serum samples from 181 healthy blood donors (91 males, 90 females) were obtained.

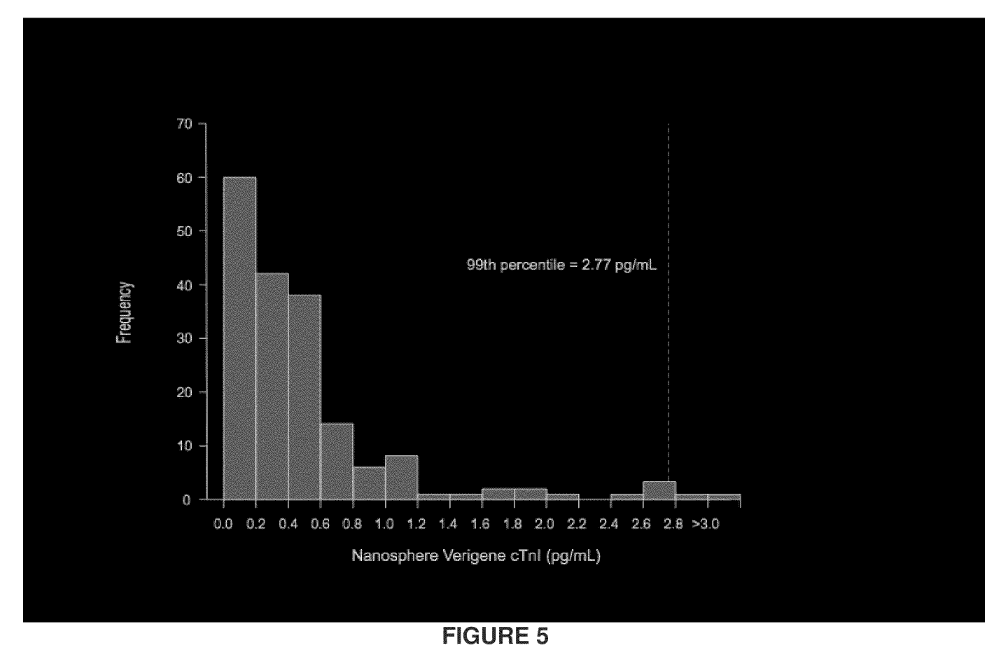

The limit of detection (LoD) of the nanoparticle-based cTnI assay, mean plus 3 SDs of 8 replicates of the zero calibrator, was 0.2 pg/mL. The linear range was 0.2 to 500 pg/mL. Imprecision (% CV) was 16.0% at 0.05 pg/mL, 9.5% at 0.5 pg/mL and 9.7% at 5 pg/mL. FIG. 5 shows that 45% of the normal subjects had a measurable concentration, with a 99th percentile of 2.8 pg/mL. The assay measured cTnI concentrations that were 1 to 2 orders of magnitude lower than the commercial assays. The last dilution prior to the >20% CV % threshold was 1.7 pg/mL at 7% CV for the nanoparticle assay, 43.3 pg/mL at 13% CV for the Siemens CS, 28.3 pg/mL at 11% CV for the Abbott Architect, 18.5 pg/mL at 13% CV for the Siemens Ultra, and 30.0 pg/mL at <1.0% CV for the Beckman Access. Correlations were excellent between assays (r=0.999 for all), but substantial differences in slopes and intercepts, respectively, were observed: Architect 2.18, 22.2; Beckman 1.34, 15.0; Siemens CS 4.03, −32.0; Siemens Ultra 5.43, −10.2.

Serum samples from ten MI patients following hospital presentation and 90 and 180 minutes after were measured by the nanoparticle-based assay and Siemens CS (99th percentile 70 pg/mL). Two (20%) and ten (100%) of the patients were cTnI positive at presentation on the CS and the nanosphere assay, respectively. Three of the patients (30%) remained cTnI negative for >180 minutes on the Siemens CS. No cross reactivity was found for skeletal troponins.

### Example 5

### Detection of Myocardial Injury in Patients With Unstable Angina (UA) Using a Nanoparticle-Based Cardiac Troponin I Assay: Observations from the PROTECT-TIMI 30 Trial

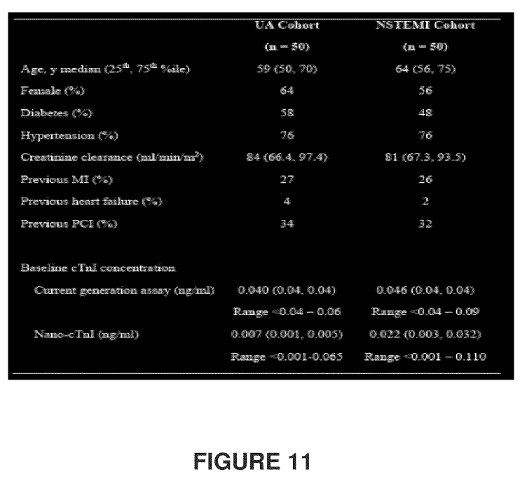

Among patients with NSTE-ACS enrolled in the PROTECT-TIMI 30 trial, a cohort of 50 patients were identified with ischemic chest pain at rest and serial negative cTnI results performed in the TIMI Biomarker Core Laboratory (Boston, Mass.) using a current commercial assay (FIG. 11). In addition, 50 patients were identified with definite myocardial injury in whom the initial current generation cTnI result was negative but results from sampling at 6-8 hours or 18-24 hours revealed a subsequent increase in cTnI.

### Methods

The nanoparticle-based assay quantifies an optical signal resulting from selective detection of the cTnI molecule through analyte-specific capture of gold nanoparticle probes, followed by subsequent signal enhancement. Briefly, capture antibody (mouse monoclonal, Nanogen) was contact-spotted in triplicate onto CodeLink activated microarray slides along with controls in ten sub-arrays and assembled into a hybridization chamber. Fifty microliters of sample was incubated for 90 minutes at 35° C. Wells were washed with 0.3% Tween 20 in phosphate-buffered saline (PBS) pH 7.4, then incubated with detection antibody (rabbit polyclonal, Nanogen) in 1% BSA, 0.3% Tween in PBS pH 7.4 and incubated for 20 minutes. Wells were washed again, then incubated in 150 pM gold nanoparticles for 10 minutes. Slides were washed, followed by a 150 mM sodium nitrate, pH 7.5 wash. Capture substrates were immersed in signal enhancement reaction mix for 9 minutes at 19° C., and rinsed. Slides were dried and scanned (see Example 1).

### Results

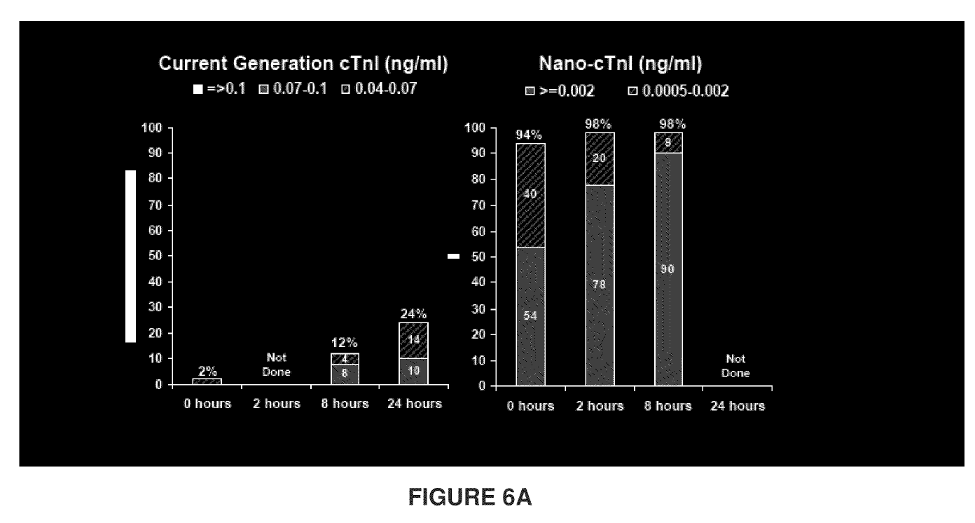

The clinical sensitivity of an ultra-sensitive nanoparticle-based cTnI assay (detection limit of 0.00015 ng/mL, referred to as nano-cTnI) was assessed in the 50 patients with UA (serial negative cTnI) and 50 patients with NSTEMI with an initially negative current generation cTnI. Measured at 0, 2 and 8 hours with the nano-cTnI assay (i.e., ultrasensitive cTnI assay), 54%, 78% and 90% of patients with unstable angina defined by a current commercial assay had an elevated nano-cTnI result (>0.002 ng/mL, 99% percentile decision-limit, CV<15%) (FIG. 6A). In patients with definite myocardial injury (current generation cTnI≧0.1 ng/mL) but an initially negative cTnI, 86% and 100% had a nano-cTnI≧0.002 ng/mL at 0 hours and 2 hour s (FIG. 6B). No patient had a positive current generation cTnI without a measurable nano-cTnI level. These data suggest that ischemia without release of troponin is in fact rare.

### Example 6

### Measurement of Ultrasensitive cTnI Levels in Patients Who Measure Below Current Generation Assays but are Detectable with the Ultrasensitive cTnI Assay

### Methods

The nanoparticle assay is based on the quantification of an optical signal resulting from selective detection of the cTnI molecule through analyte-specific capture of gold nanoparticle probes, followed by subsequent signal enhancement. Briefly, capture antibody (mouse monoclonal, Nanogen) was contact-spotted in triplicate onto CodeLink activated microarray slides along with controls in ten sub-arrays and assembled into a hybridization chamber. Fifty microliters of sample was incubated for 90 minutes at 35° C. Wells were washed with 0.3% Tween 20 in phosphate-buffered saline (PBS) pH 7.4, then incubated with detection antibody (rabbit polyclonal, Nanogen) in 1% BSA, 0.3% Tween in PBS pH 7.4 and incubated for 20 minutes. Wells were washed again, then incubated in 150 pM gold nanoparticles for 10 minutes. Slides were washed, followed by a 150 mM sodium nitrate, pH 7.5 wash. Capture substrates were immersed in signal enhancement reaction mix for 9 minutes at 19° C., and rinsed. Slides were dried and scanned (see Example 1).

### Results

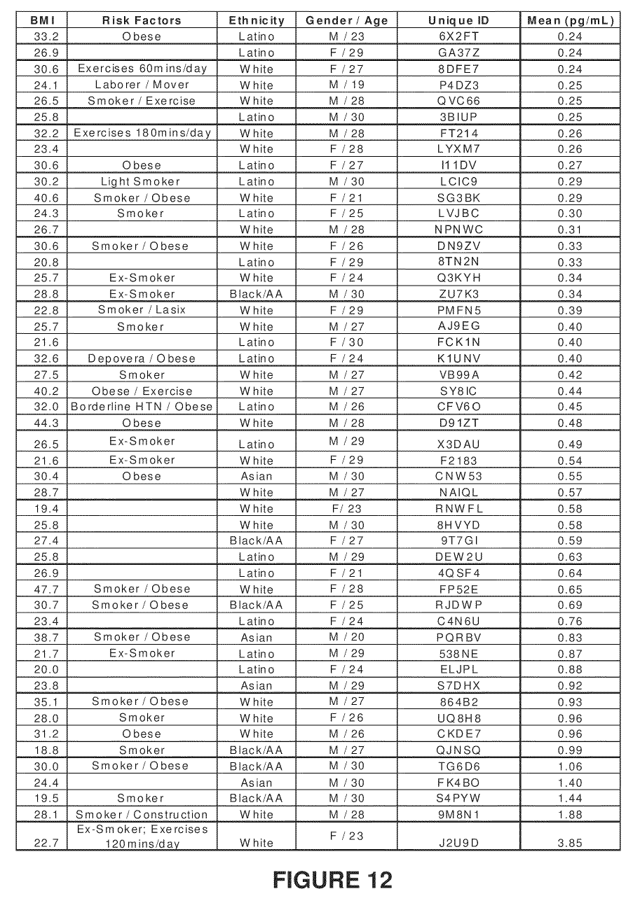

FIG. 12 shows troponin measurements using the ultrasensitive cTnI assay for 50 “normal” samples. These 50 samples had measurable ultrasensitive cTnI levels (i.e. <10 pg/mL) that were above the limit of detection of the ultrasensitive cTnI assay (limit of detection of 200 fg/mL). The risk factors which may lead to an increased cTnI level in the ultrasensitive range are listed in a separate column of the chart. For example, many of the tested patients that had measurable ultrasensitive cTnI levels were smokers or former smokers. Additionally, many had a very high body mass index (BMI). These cTnI levels may be associated with earlier and more minor cardiac events that become detectable with the ultrasensitive cTnI assay.

### Example 7

### Detection of Rat Troponin I

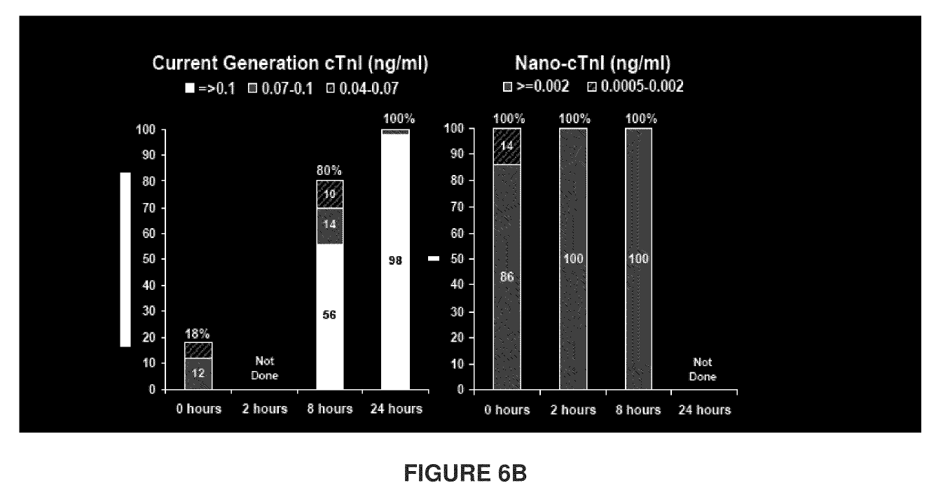

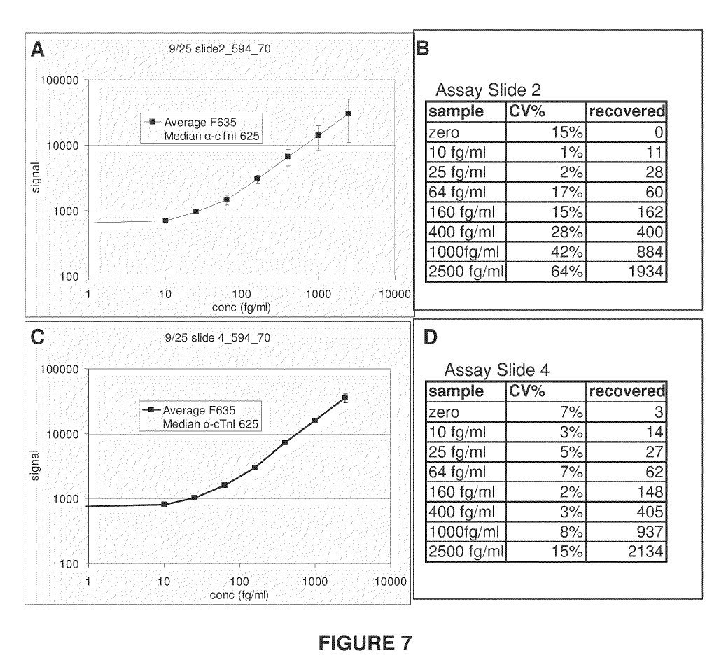

In addition to measurements of human cTnI, ultrasensitive measurements of cTnI in rats could benefit cardiac toxicity studies associated with drug development via earlier diagnosis of drug toxicity (Gould et al., J. Natl. Cancer Inst., 99:1724 (2007)). Additional data collected using rat troponin I as an antigen demonstrates that even lower concentrations of rat troponin I are quantifiable when using the nanoparticle assay format. In this example, monoclonal anti-troponin I antibodies (Hytest clone # 625) were immobilized on Hydrogel coated glass substrates using microarray printing methods well known in the art. Following washing of the substrates to remove excess antibody, the target troponin antigen was incubated in 1% rat serum on the slide surface, followed by labeling with a biotinylated rabbit anti-troponin antibody in a sandwich assay format. The sandwich complex was further labeled with streptavidin, followed by binding of 13 nm diameter gold particles with covalently immobilized biotins attached to the surface. Detection of the labeled troponin was achieved by catalytically reducing silver onto the surface of the gold particles followed by imaging light scattered by the silver amplified gold particles (Storhoff et al. (Biosens. & Bioelectron., 19:875 (2004)). In the example shown in FIG. 7, the mean signal intensity was measured as a function of rat cTnI concentration by adding known amounts of rat troponin I (10-2500 fg/mL) into the rat serum. This example demonstrates that with the described assay a limit of detection of 10 fg/mL ratcTnI is achieved with linearity up to 400 fg/mL, and further dose response up to 2500 fg/mL.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method to determine a test subject at risk of or suspected of having unstable angina or non-ST elevation myocardial infarction, comprising:
a) detecting the amount of first complexes formed by contacting a first physiological fluid sample from a test subject at risk of or suspected of having acute coronary syndrome and a solid substrate having one or more cardiac troponin-specific antibodies, thereby detecting the concentration of troponin in the first physiological sample; and
b) comparing the amount of the first complexes with second complexes formed by contacting a second physiological sample from the test subject from a different time point with a solid substrate having the one or more cardiac troponin-specific antibodies, wherein the rate of increase in cardiac troponin concentrations over time is indicative of unstable angina or non-ST elevation myocardial infarction.

2. A method to detect cardiac troponin concentrations of 10 pg/mL or less, comprising:
a) detecting the amount of first complexes formed by contacting a first physiological fluid sample from a test subject and a solid substrate having one or more cardiac troponin-specific antibodies, thereby detecting the concentration of troponin in the first physiological sample, wherein cardiac troponin concentrations of 10 pg/mL or less are detectable.

3. The method of claim 2 further comprising comparing the amount of the first complexes with second complexes formed by contacting a second physiological sample from the test subject from a different time point with a solid substrate having the one or more cardiac troponin-specific antibodies, wherein the rate of increase in the amount of cardiac troponin over time is indicative of acute coronary syndrome in the test subject.

4. The method of claim 1 wherein cardiac troponin concentrations from 5 fg/mL to 10 pg/mL are detectable.

5. The method of claim 1 wherein the first complexes are detected by contacting one or more different cardiac troponin-specific antibodies linked to a detectable molecule with the first complexes.

6. The method of claim 5 wherein the detectable molecule is a gold nanoparticle.

7. A method to detect cardiac troponin levels in a physiological sample comprising:
a) providing a mixture comprising a physiological fluid sample from a test subject and a solid substrate having one or more cardiac troponin-specific antibodies so as to form a first complex;
b) contacting the complex with one or more different cardiac troponin-specific antibodies bound to a detectable molecule; and
c) detecting the detectable molecule, thereby detecting cardiac troponin levels, wherein cardiac troponin levels from 5 fg/mL to 10 pg/mL are detectable.

8. The method of claim 7 further comprising comparing the amount of the detectable molecule to the amount of detectable molecule obtained by contacting the one or more different cardiac troponin-specific antibodies bound to the detectable molecule with complexes formed by contacting a second physiological sample from the test subject from a different time point with a solid substrate having the one or more cardiac troponin-specific antibodies.

9. The method of claim 1 wherein the one or more cardiac troponin-specific antibodies are specific for one epitope of troponin.

10. The method of claim 1 wherein the one or more cardiac troponin-specific antibodies bind more than one epitope of troponin.

11. The method of claim 1 wherein the solid substrate comprises different cardiac troponin-specific antibodies physically separated on the solid substrate.

12. The method of claim 11 wherein the different antibodies are specific for different epitopes of cardiac troponin, thereby allowing for independent detection of two or more different cardiac troponin epitopes in the physiological sample.

13. The method of claim 12 wherein the independent detection of two or more different cardiac troponin epitopes in the physiological sample allows for differentiation of cardiac events including unstable angina, non-ST elevation myocardial infarction, or acute myocardial infarction.

14. The method of claim 1, 2 or 7 wherein the sample is a blood sample.

15. The method of claim 7 wherein cardiac troponin levels are indicative of a test subject not at risk of having acute coronary syndrome.

16. The method of claim 15 wherein the levels of troponin in the test subject not at risk of acute coronary syndrome are less than about 300 fg/mL.

17. A computerized method of detecting acute coronary syndrome, the computerized method comprising:
receiving first and second inputs indicative of detected amounts of cardiac troponin concentrations by contacting respective first and second physiological fluid samples taken at respective first and second time points from a test subject with respective first and second solid substrates, each substrate having one or more cardiac troponin-specific antibodies, wherein cardiac troponin concentrations 10 pg/mL or less are detectable;
evaluating the first and second inputs as a function of one or more stored algorithms to identify one or more cardiac conditions of the test subject; and
providing an output indicative of the one or more identified cardiac conditions of the test subject.

18. A computer-readable medium, with instructions thereon, which when executed by a processor of a computing device, cause the computing device to:
receive first and second inputs indicative of detected amounts of complexes in first and second physiological fluid samples taken at respective first and second time points from a test subject;
evaluate the first and second inputs as a function of one or more algorithms stored on the computer-readable medium to identify one or more cardiac conditions of the test subject; and
provide an output indicative of the one or more identified cardiac conditions of the test subject.
